(19) United States
(12) Patent Application Publication
(10) Pub. No.: US 2009/0299077 A1
(43) Pub. Date: Dec. 3, 2009

(54) Salts of (R)-5-(2phenylsulphonylethenyl)-3-(N-methylpyrrolidin-2-ylmethyl)-1H-indole, 5-bromo-3-[(R)-1-methyl-pyrrolidin-2-ylmethyl]-1H-indole and of eletriptan

(76) Inventors: Vinod Kumar KANSAL, Haryana (IN); Dhirenkumar N. MISTRY, Gujarat (IN); Rakesh Ravjibhai PATEL, Greater Noida (IN); Saurabh PANDEY, Greater Noida (IN)

Correspondence Address: KENYON & KENYON LLP, ONE BROADWAY, NEW YORK, NY, 10004, US

(21) Appl. No.: 12/454,806

(22) Filed: May 22, 2009

Related U.S. Application Data
(60) Provisional application No. 61/128,659, filed on May 22, 2008.
(60) Provisional application No. 61/080,853, filed on Jul. 15, 2008.
(60) Provisional application No. 61/137,244, filed on Jul. 28, 2008.

(51) Int. Cl. C07D 403/06

(52) U.S. Cl. 548/468

(57) ABSTRACT

The present invention relates to salts of (R)-5-(2-phenylsulphonylethenyl)-3-(N-methylpyrrolidin-2-ylmethyl)-1H-indole of the formula:

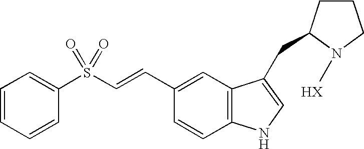

wherein HX is an acid selected from para-toluene sulfonic acid, benzene sulphonic acid, trifluoroacetic acid, methane sulphonic acid, formic acid and succinic acid; and to processes of preparing and using such salts.

### CROSS REFERENCE TO RELATED APPLICATIONS

The present invention claims the benefit of the following U.S. Provisional Patent Application Nos. 61/128,659, filed May 22, 2008; 61/137,244, filed Jul. 28, 2008; and 61/080,853, filed Jul. 15, 2008. The contents of these applications are incorporated herein by reference.

### FIELD OF INVENTION

The present invention relates to salts of Eletriptan and its precursors (R)-5-(2-phenylsulphonylethenyl)-3-(N-methylpyrrolidin-2-ylmethyl)-1H-indole (“EPSIP”) and 5-bromo-3-[(R)-1-methyl-pyrrolidin-2-ylmethyl]-1H-indole (“BIP”), and processes for preparing them.

### BACKGROUND OF THE INVENTION

Eletriptan (“ELT”), 3-[[(R]-1-methyl-2-pyrrolidinyl)methyl]-5-[2-(phenyl-sulfonyl)ethyl]indole, having the following formula:

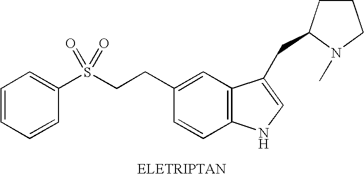

is used for the acute treatment of migraine with or without aura in adults. Eletriptan is a selective 5-hydroxytryptamine 1B/1D receptor agonist, which is administrated as eletriptan hydrobromide (“ELT-HBr”). Eletriptan tablets are marketed by Pfizer under the name RELPAX®.

Eletriptan, and intermediates thereof, are described in U.S. Pat. Nos. 5,545,644 and 5,607,951, and in EP 592438. These patents report processes for forming indoles by transition metal catalyzed cyclization of a dihalogenated intermediate and by alkylation with alkyl halides in the presence of a base. These processes also report the preparation of Eletriptan Base and hemisuccinate salt thereof.

U.S. Pat. No. 7,288,662 reports a process for the preparation of Eletriptan hydrobromide salt, an intermediate and dimer free products.

The present invention addresses the need for the preparation of pure Eletriptan HBr via its salt form and via the salts of its precursors (R)-5-(2-phenylsulphonylethenyl)-3-(N-methylpyrrolidin-2-ylmethyl)-1H-indole (“EPSIP”) and 5-bromo-3-[(R)-1-methyl-pyrrolidin-2-ylmethyl]-1H-indole (“BIP”).

### SUMMARY OF THE INVENTION

In one embodiment, the invention comprises a salt of (R)-5-(2-phenylsulphonyl-ethenyl)-3-(N-methylpyrrolidin-2-ylmethyl)-1H-indole (“EPSIP”) of the formula:

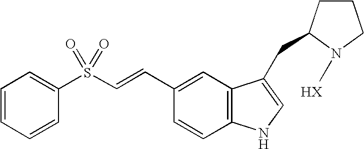

wherein HX is an acid selected from a group consisting of para-toluene sulfonic acid (“PTSA”), benzene sulphonic acid, trifluoroacetic acid (“TFA”), methanesulphonic acid (“MSA”), formic acid, and succinic acid.

In another embodiment, the invention comprises the preparation of an EPSIP salt selected from a PTSA salt, a benzene sulphonic acid salt, a TFA salt, a MSA salt, a formic acid salt, and a succinic acid salt, the preparation comprising reacting EPSIP with an acid selected from PTSA, benzene sulphonic acid, TFA, MSA, formic acid and succinic acid.

In yet another embodiment, the invention comprises the use of the above EPSIP salt for the preparation of Eletriptan and salts thereof of formula:

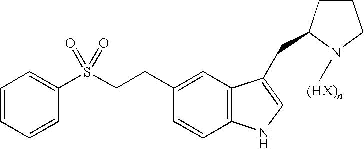

wherein n is 0 or 1.

In one embodiment, the invention comprises a process for preparing Eletriptan and salt thereof comprising preparing any of the above EPSIP salts according to the processes of the present invention, and converting them to Eletriptan and salts thereof.

In one embodiment the invention comprises a PTSA salt of Eletriptan (“ELT-PTSA”) of formula VII:

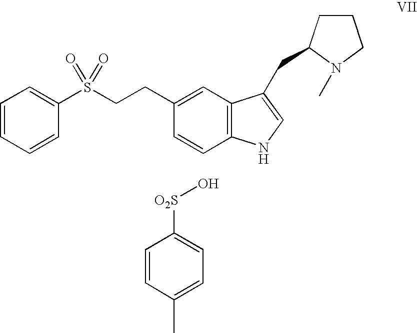

In another embodiment, the invention comprises the preparation of ELT-PTSA comprising reacting ELT with PTSA.

In yet another embodiment, the invention comprises the use of ELT-PTSA for the preparation of Eletriptan HBr

In another embodiment, the invention provides a process for the preparation of eletriptan HBr comprising preparing ELT-PTSA according to the process of the present invention, and converting it to eletriptan HBr.

In one embodiment, the invention comprises a salt of 5-bromo-3-[(R)-1-methyl-pyrrolidin-2-ylmethyl]-1H-indole (“BIP”) of the following formula:

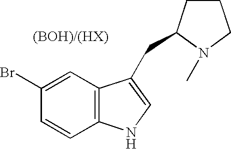

wherein B is potassium, and HX is an acid selected from oxalic acid, succinic acid, and fumaric Acid.

In another embodiment, the invention comprises the preparation of a BIP salt comprising reacting BIP with an acid selected from oxalic acid, succinic acid and fumaric acid or with potassium hydroxide, wherein the obtained salt is oxalate, succinate, fumarate or potassium.

In yet another embodiment, the invention comprises the use of the above BIP salt for the preparation of Eletriptan and salt thereof.

In another embodiment, the present invention provides a process for the preparation of eletriptan and salts thereof comprising preparing the above BIP salts according to the process of the present invention, and converting them to eletriptan and salts thereof.

### DETAILED DESCRIPTION OF THE INVENTION

The present invention provides a method to purify Eletriptan and its intermediates, (R)-5-(2-phenylsulphonylethenyl)-3-(N-methylpyrrolidin-2-ylmethyl)-1H-indole (“EPSIP”) and 5-bromo-3-[(R)-1-methyl-pyrrolidin-2-ylmethyl]-1H-indole (“BIP”).

When these intermediates are prepared and purified according to prior art methods (e.g., according to comparative examples 19-23 and (33), they are significantly more contaminated and thus negatively affect the purity of eletriptan HBr. However, when using the method of the present invention to purify these intermediates the final product is significantly purer.

The purification method of the present invention comprises the conversion of the non-ionic starting form of these intermediates (i.e. EPSIP and BIP) to an ionic form, which is an acidic or basic salt form.

The present invention comprises a salt of (R)-5-(2-phenylsulphonylethenyl)-3-(N-methylpyrrolidin-2-ylmethyl)-1H-indole (“EPSIP”) of the following formula:

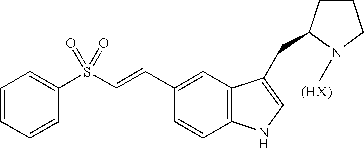

wherein HX is an acid selected from PTSA, benzene sulphonic acid, TFA, MSA, formic acid and succinic acid.

According to some embodiments, the above salts of EPSIP are provided in an isolated form.

As used herein, the term “isolated” in reference to the EPSIP salt corresponds to EPSIP salt that is physically separated from the reaction mixture in which it is prepared. The separation can be done, for example, by filtering precipitated EPSIP salt.

The above EPSIP salt can be prepared by a process comprising reacting EPSIP with an acid selected from PTSA, benzenesulphonic acid, TFA, MSA, formic acid and succinic acid.

The reaction comprises: a) dissolving EPSIP in a solvent to obtain a solution, b) combining said solution with an acid selected from the group consisting of PTSA, benzene sulphonic acid, TFA, MSA, formic acid and succinic acid, and c) precipitating EPSIP salt to obtain a suspension.

According to one embodiment, the acid is MSA.

According to an embodiment, EPSIP is dissolved in an organic solvent. According to some embodiments the organic solvent is selected from the group consisting of ketones, alcohols, nitrites, and combinations thereof. According to some embodiments, the ketone is a C₁₋₄ ketone such as acetone or methylethylketone (MEK). According to some embodiments, the alcohol is a C₁₋₄ alcohol, such as methanol, ethanol, propanol or isopropanol. According to some embodiments, the nitrile is a C₁₋₃ nitrile, such as acetonitrile or propionitrile. According to a preferred embodiment, the solvent is ethanol or acetonitrile.

According to some embodiments, dissolving EPSIP is achieved by heating. According to some embodiments, the heating is done to a temperature from about 30° C. to about 65° C.

The EPSIP solution is combined with an acid selected from PTSA, benzene sulphonic acid, TFA, MSA, formic acid and succinic acid to obtain a suspension. According to some embodiments, the acid is added to the EPSIP solution at a temperature from about 30° C. to about 65° C.

When formic acid is added, the addition is followed, in some embodiments, by an addition of ammonium formate salt.

In some embodiments, the addition of the acid results in the precipitation of the EPSIP salt. In other embodiments, the addition of the acid results in a soluble salt that may be precipitated by cooling the solution. According to some embodiments, cooling is to a temperature from about 0° C. to about 20° C. Optionally, cooling can be preformed gradually.

In some embodiments, the suspension is stirred. Stirring can be done, for example, for a period of about 0.5 hour to about 3 hours or for a period of about 0.5 hour to about 1 hour.

Typically, the precipitated salt can be recovered from the suspension. The recovery can be done, for example by filtering the suspension.

The obtained EPSIP salts have a purity of at least 96% area by HPLC. In some embodiments, the obtained EPSIP salts have a purity from about 96% to about 99.1% area by HPLC. This obtained purity is significantly greater than the purity of about 88% area by HPLC that is obtained when EPSIP is purified according to a prior art process.

The purified EPSIP salt may be converted to eletriptan and salts thereof, as exemplified in example 15.

The present invention also relates to the PTSA salt of Eletriptan (“ELT-PTSA”), its preparation and its use to prepare Eletriptan and the HBr salt of Eletriptan.

The preparation of the PTSA salt serves as a means to obtain a pure intermediate of ELT (see example 16 compared to example 23), where the purity of ELT base that is obtained from ELT PTSA is 99.75% as compared to 52.6% when obtained using the prior art process.

In one embodiment the present invention encompasses the PTSA salt of Eletriptan of formula VII:

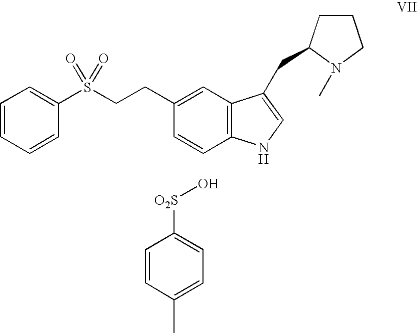

The ELT-PTSA salt may be provided in an isolated form. According to some embodiments, the isolated PTSA-ELT salt is solid, and according to some embodiments, it is crystalline. As used herein, the term “isolated” in reference to ELT-PTSA salt corresponds to Eletriptan-PTSA salt that is physically separated from the reaction mixture. The separation can be done, for example, by filtration. ELT-PTSA can be prepared by a process comprising reacting ELT base with PTSA.

In the present invention, ELT base is not isolated (see examples 6 and 15), and thus ELT base is reacted in situ with PTSA to precipitate the ELT-PTSA salt.

In one embodiment, ELT-PTSA salt can be used to prepare ELT-HBr of formula I:

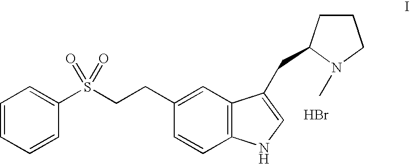

The process comprises reacting ELT-PTSA with a base and then with HBr to obtain ELT-HBr.

The process can be described in the following scheme:

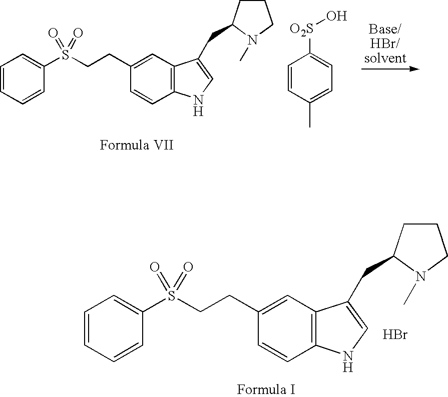

Usually, ELT-PTSA is first converted to ELT base, which is then reacted with HBr, providing ELT-HBr. ELT base can be isolated prior to reacting with HBr, or it can be reacted in situ with HBr. According to one embodiment, ELT base is not isolated prior to the conversion to ELT-HBr.

Preferably, HBr is present in the form of a gas or in a solution. Typically, HBr is dissolved in a solvent, such as iso-propanoic acid or isobutyric acid.

In some embodiments, the reaction is done in a solvent. According to some embodiments, the solvent is selected from a C₃-C₅ ketone, such as acetone or MEK, a C₃-C₅ ether, such as dimethoxyethane or THF, and combinations thereof.

The conversion of ELT-PTSA to ELT base may be carried out in an aqueous base such as an aqueous solution of ammonia.

In some embodiments, the reaction of the ELT base and HBr provides a precipitate of ELT-HBr. The obtained ELT-HBr salt can be recovered from the mixture, for example, by filtration.

The ELT HBr that is obtained according to the process of the present invention has a purity of at least 97.9% area by HPLC. According to some embodiments, the obtained ELT HBr has a purity from about 97.9% to about 99.78% area by HPLC.

The present invention also relates to salts of 5-bromo-3-[(R)-1-methyl-pyrrolidin-2-ylmethyl]-1H-indole (“BIP”), which is the starting material of EPSIP. The BIP salts provided by the present invention have the following formula:

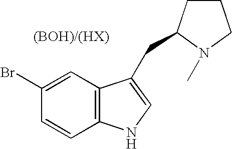

wherein B is potassium, and HX is an acid selected from oxalic acid, succinic acid and fumaric acid. Preferably, the above salts of BIP are provided in an isolated form.

As used herein, the term “isolated” in reference to the BIP salt refers to BIP salt that is physically separated from the reaction mixture in which it is prepared. Separation can be done, for example, by filtering precipitated BIP salt.

Typically, crude BIP as obtained in the prior art comprises three main impurities of the following formulas,

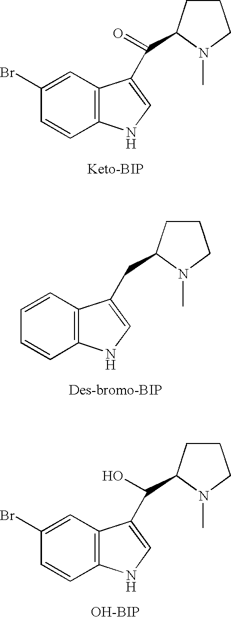

as exemplified in example 29. These impurities are difficult to remove by conventional purification methods. However, all three of these problematic impurities are removed efficiently by the purification method of the present invention.

The method comprises reacting BIP with an acid selected from oxalic acid, succinic acid, and fumaric acid or with potassium hydroxide.

The reaction comprises a) dissolving BIP to obtain a solution, and b) combining said solution with an acid selected from oxalic acid, succinic acid, and fumaric acid or with potassium hydroxide to obtain a suspension.

Ordinarily in this method, BIP is dissolved in an organic solvent. According to one embodiment, the organic solvent is an alcohol. Preferably, the alcohol is C₁₋₄ alcohol, such as methanol, ethanol, propanol or isopropanol.

Dissolving BIP in an organic solvent may be achieved, for example, by heating. According to some embodiments, the heating is done to a temperature from about 30° C. to about 60° C. According to some embodiments, the heating is done to a temperature from about 35° C. to about 45° C.

The solution is combined with an acid selected from oxalic acid, succinic acid, and fumaric acid or with potassium hydroxide (base) to obtain a suspension. Typically, the acid or base is added at the temperature to which the BIP solution had been heated.

When oxalic acid is used, it may be added, for example, in a dihydrate form in a methanol solution. The suspension may be stirred with gradual cooling. The stirring of the suspension may be done, for example from about 0.5 hour to about 3 hours, or from about 0.5 hour to about 1.5 hours, or form about 0.5 hour to about 1 hour. Gradual cooling may be carried out by removing the heat source and allowing the mixture to cool in an ambient or room temperature environment, for example at a rate of from about 1 degree per minute to about 5 degrees per minute, or from about 1 degree per minute to about 3 degrees per minute, or from about 1 degree per minute to about 2 degrees per minute.

The precipitated BIP salt can be recovered, for example, by filtering the suspension to obtain the desired salt. According to some embodiments, the filtration is performed at a temperature from about 5° C. to about 25° C.

The purified BIP salt may be converted to eletriptan and salts thereof as exemplified in examples 29 to 32.

### EXAMPLES

The HPLC method for Eletriptan Base, Eletriptan PTSA, Eletriptan HBr, EPSIP, EPSIP salts and Acetyl EPSIP is described below.

### Chromatographic Conditions:

| Column: | XTerra RP8, 3.5μ, 150 × 4.6 mm, | |
|---|---|---|
| Flow | 1.5 | mL/min |
| Injection Volume | 10 | μl |
| Detector | 220 | nm |
| Column temperature | 30° | C. |
| Sample temperature | 10° | C. |

| | |
|---|---|
| Diluent | Eluent A:Eluent B (1:1) |

### Gradient Program:

| Time (min) | % Eluent A | % Eluent B |
|---|---|---|
| 0 | 90 | 10 |
| 12 | 75 | 25 |
| 25 | 50 | 50 |
| 32 | 20 | 80 |
| 35 | 90 | 10 |
| 40 | 90 | 10 |

The components of the mobile phase were Eluent A, which is a buffer, and Eluent B which is acetonitrile. The buffer was prepared as follows: Prepare 0.02M potassium dihydrogen phosphate in water. Adjust the pH to 3.0 using 10% v/v orthophosphoric acid.

### The HPLC Method for BIP and BIP Salt is Described Below:

Chromatographic conditions

| Column: | XBridge C18, 3.5μ, 150 × 4.6 mm | |
|---|---|---|
| Flow | 1.5 | ml/min |
| Injection Volume | 10 | μl |
| Detector | 220 | nm |
| Column temperature | 30° | C. |

| | |
|---|---|
| Diluent | Eluent A:Eluent B (1:1) |

### Gradient Program:

| Time (min) | % Eluent A | % Eluent B |
|---|---|---|
| 0 | 90 | 10 |
| 12 | 75 | 25 |
| 25 | 50 | 50 |
| 30 | 20 | 80 |
| 35 | 90 | 10 |
| 40 | 90 | 10 |

The components of the mobile phase were Eluent A, which is a buffer, and Eluent B which is acetonitrile. The buffer was prepared as follows: Prepare 0.02M potassium dihydrogen phosphate in water. Adjust the pH to 3.0 using 10% v/v orthophosphoric acid.

Water content for Eletriptan HBr and EPSIP salts was determined by Karl Fischer analysis (USP,29<921>).

The melting range for Eletriptan HBr and EPSIP salts was determined using a melting point apparatus and melting points are uncorrected.

### Example 1

### Preparation of N-Acetyl EPSIP

(a) (R)-5-Bromo-3-(N-methylpyrrolidine-2-ylmethyl)-1H-Indole (100 g, 0.341 mol), dimethylformamide (DMF) (200 mL), triethylamine (TEA) (69.3 mL), 4-dimethyl-aminopyridine (DMAP)(0.083 g, 0.02 mol) and acetic anhydride (48.7 g) were charged to a dry 4-neck round bottom flask. The reaction mixture was heated to 100-105° C. for 2-3 hours.

(b) A mixture of DMF (200 ml), palladium acetate (3.06 g, 0.0136 mol) and tri-o-tolylphosphine (12.45 g, 0.0409 mol) was stirred under argon gas purging for 1 hour. Phenyl vinyl sulphone (63.19 g, 0.375 mol), N,N-diisopropylamine (71.4 mL, 0.4092 mol) and charged part (a) previously prepared solution and raise the temperature up to 90-95° C. for 3-4 hours. The reaction mixture was then cooled to 20-25° C., and a solution of 30% aqueous hydrochloric acid (HCl) (200 mL) was added over 30-45 min. The resulting mixture was filtered to remove the catalyst and a further 200 ml methanol and get pH 8-9 using 50% w/w aqueous sodium hydroxide solution were added to the filtrate to precipitate the product. The resulting suspension was filtered and the filtered product was washed with mixture of methanol:water (1:4) to yield crude R-1-acetyl-5-(2-phenyl-sulphonylethenyl)-3-(N-methylpyrrolidin-2-ylmethyl)-1H-indole). Dry wt (140 g, 97.2% yield, 78.2% purity).

### Purification 1:

Crude (R)-1-acetyl-5-(2-phenylsulphonylethenyl)-3-(N-methylpyrrolidin-2-ylmethyl)-1H-indole was stirred in methanol (600 mL) for 1-2 hours and then filtered to provide pure (R)-1-acetyl-5-(2-phenylsulphonylethenyl)-3-(N-methylpyrrolidin-2-ylmethyl)-1H-indole (90 g dry, 62.5% yield, 98.5% purity).

### Purification 2:

Crude (R)-1-acetyl-5-(2-phenylsulphonylethenyl)-3-(N-methylpyrrolidin-2-ylmethyl)-1H-indole (40 g, 0.0947 mol) was dissolved in acetone (80 mL) at 60° C. and was charged water (96 mL) slowly. The product precipitated out and was filtered after 2 hours to provide pure (R)-1-acetyl-5-(2-phenylsulphonylethenyl)-3-(N-methylpyrrolidin-2-ylmethyl)-1H-indole (26 g dry, 65% yield, 90% purity).

### Example 2

### Preparation of Acetyl Eletriptan from Acetyl EPSIP

The following: (R)-1-acetyl-5-(2-phenylsulphonylethenyl)-3-(N-methylpyrrolidin-2-ylmethyl)-1H-indole (20 g, 0.0473 mol), acetone (300 mL), water (30 mL) were added to a 4 neck round bottom flask (RBF). The mixture was cooled to 8-10° C. To the cooled mixture was added methanesulphonic acid (8.08 g, 0.0842 mol). The resulting reaction mixture was then transferred to a hydrogenation vessel. To this was added 10% palladium on carbon (5 g dry basis) and water (10 mL). The resulting mixture was hydrogenated under an atmosphere of hydrogen gas for 2-3 hours. The reaction mixture was then filtered to remove the catalyst. The acetone was distilled out of the filtrate. The remaining filtrate was diluted with water (200 mL) and washed by methyl t-butyl ether (100 mL). The pH of the aqueous layer was adjusted to 9-10 using an aqueous ammonia solution. The product precipitated and was filtered after 1 hour. The filtered product was washed with water to provide (R)-1-acetyl-5-(2-phenylsulphonylethyl)-3-(N-methyl-pyrrolidin-2-ylmethyl)-1H-indole (21.3 g dry, 95% yield, 95% purity).

### Example 3

### Preparation of Eletriptan-PTSA Salt from Acetyl EPSIP

(R)-1-Acetyl-5-(2-phenylsulphonylethyl)-3-(N-methylpyrrolidin-2-ylmethyl)-1H-indole (20 g, 0.047 mol), acetone (200 mL), methanol (20 mL), and potassium carbonate (9.76 g, 0.0707 mols) were added to a 4 neck RBF at 20-25° C. The mixture was stirred for 6-7 hours. To the mixture was added carbon (10 g). After 30 min, the mixture was filtered through bed of diatomaceous earth. The filtrate was concentrated, followed by addition of acetone (50 ml) and again concentrated to provide Eletriptan base as an oil. To the eletriptan base was added acetone (100 mL), and to this mixture was added a solution of p-toluene sulphonic acid (9.9 g dissolved in 40 mL acetone) at 35-40° C. After 2-3 hours, this mixture was filtered and the recovered solid was washed with acetone to provide Eletriptan-PTSA salt (15.6 g, 60% yield, 98.96% purity).

### Example 4

### Preparation of N-Acetyl EPSIP

(a) (R)-5-Bromo-3-(N-methylpyrrolidine-2-ylmethyl)-1H-Indole (100 g, 0.341 mol), DMF (200 mL), TEA (69.3 mL) and acetic anhydride (48.7 g) were added to a dry 4-neck RB flask. The reaction mixture was heated to 100-105° C. for 2-3 hours.

(b) A mixture of DMF (200 ml), palladium acetate (3.06 g, 0.0136 mol) and tri-o-tolylphosphine (12.45 g, 0.0409 mol) was stirred under argon gas purging for 1 hour. Phenyl vinyl sulphone (63.19 g, 0.375 mol), N,N-di-isopropylamine (71.4 mL, 0.4092 mol) and charged part (a) previously prepared solution and raised the temperature to 90-95° C. for 3-4 hours. The reaction mixture was then cooled to 20-25° C. and a solution of 30% aqueous HCl (200 mL) was added over 30-45 min. The resulting mixture was filtered to remove the catalyst and a further 200 ml methanol and get pH 8-9 using 50% w/w aqueous sodium hydroxide solution were added to the filtrate to precipitate the product. The resulting suspension was filtered and the thus recovered solid was washed with a mixture of methanol:water (1:4) to yield crude R-1-acetyl-5-(2-phenylsulphonylethenyl)-3-(N-methylpyrrolidin-2-ylmethyl)-1H-indole). Dry wt (140 g, 97.2% yield, 78.2% purity).

### Purification 1:

Crude (R)-1-acetyl-5-(2-phenylsulphonylethenyl)-3-(N-methylpyrrolidin-2-ylmethyl)-1H-indole was stirred in methanol (600 mL) for 1-2 hr. This mixture was then filtered to provide pure (R)-1-acetyl-5-(2-phenylsulphonylethenyl)-3-(N-methylpyrrolidin-2-ylmethyl)-1H-indole (90 g dry, 62.5% yield, 98.5% purity).

### Purification 2:

Crude (R)-1-acetyl-5-(2-phenylsulphonylethenyl)-3-(N-methylpyrrolidin-2-ylmethyl)-1H-indole (40 g, 0.0947 mol) was dissolved in acetone (80 mL) at 60° C. and charged water (96 mL) slowly. The product precipitated and after 2 hours the mixture was filtered to give pure (R)-1-acetyl-5-(2-phenylsulphonylethenyl)-3-(N-methylpyrrolidin-2-ylmethyl)-1H-indole (26 g dry, 65% yield, 90% purity)

### Example 5

### Preparation of EPSIP (Deacetylation of EPSIP)

(R)-1-Acetyl-5-(2-phenylsulphonylethenyl)-3-(N-methylpyrrolidin-2-ylmethyl)-1H-indole) (30 g, 0.071 mol) was dissolved in a mixture of methanol (300 mL) and water (600 mL) at 20-25° C. in a 4-neck RBF. Potassium carbonate (9.89 g, 0.071 mols) was added and the resulting reaction mass was stirred at 40° C. for 1 hour. Carbon (3 g) was added to the mixture. The resulting mixture was filtered through a bed of diatomaceous earth. The filtrate was diluted by the addition of water (300 mL) at 60° C. over a period of 1 hour. The reaction mixture was then cooled to 20-25° C. and diluted by adding water (420 mL). The resulting mixture was stirred for 2 hours. The product was collected by filtration and washed with (methanol:water-1:2) to provide (R)-5-(2-phenylsulphonylethenyl)-3-(N-methylpyrrolidin-2-ylmethyl)-1H-indole (25.6 g dry, 94.8% yield, 98.2% purity).

### Example 6

### Preparation of Eletriptan-PTSA

(R)-5-(2-Phenylsulphonylethenyl)-3-(N-methylpyrrolidin-2-ylmethyl)-1H-indole (20 g, 0.0526 mol), acetone (200 mL) and water (30 mL) were added to a 4 neck RBF and the mixture was cooled to 8-10° C. To the cooled mixture was added methanesulphonic acid (8.08 g, 0.0842 mol). The resulting reaction mixture was then transferred to a hydrogenation vessel. To the mixture was added 10% palladium on carbon (5 g dry basis). This mixture was hydrogenated under an atmosphere of hydrogen gas for 2-3 hours. The reaction mixture was then filtered to remove the catalyst. The filtrate was concentrated to remove the acetone and the concentrate was diluted with water (200 mL) and washed with methyl tert-butyl ether (MTBE) (100 mL). Adjusted the pH of the aqueous layer to pH 12 using an aqueous ammonia solution, and then extracted eletriptan base with MTBE (200 mL). The aqueous layer was washed with MTBE (200 mL). The MTBE was removed by distillation, acetone (100 mL) was added and the mixture was again concentrated to provide eletriptan base as a residue. The residue was dissolved in acetone (60 mL) at 40° C. To this solution was added a p-toluene sulphonic acid solution (9.9 g, 0.0521 mols dissolved in 40 mL acetone) to get pH 4.5-5.0. After 2.0 hr the product was collected by filtration and washed with acetone (20 mL) at 20-25° C. to provide eletriptan-PTSA salt (20.6 g dry, 73.5% yield, 98.8% purity).

### Example 7

### Preparation of N-Acetyl EPSIP

(a) (R)-5-Bromo-3-(N-methylpyrrolidine-2-ylmethyl)-1H-indole (100 g, 0.341 mol), DMF (200 mL), TEA (69.3 mL), DMAP (1.0 g, 0.008 mol) and acetic anhydride (48.7 g) were added to a dry 4-neck RB flask. The resulting mixture was heated to 100-105° C. for 2-3 hr.

(b) A mixture of DMF (200 mL), palladium acetate (4.59 g, 0.0204 mol) and tri-o-tolylphosphine (18.68 g, 0.0613 mol) was stirred under argon gas purging for 1 hr. Phenyl vinyl sulphone (63.19 g, 0.375 mol), N,N-diisopropyl amine (71.4 mL, 0.4092 mol) and charged part (a) previously prepared solution. The resulting mixture was heated to 90-95° C. for 3-4 hr. The reaction mixture was then cooled to 20-25° C. The reaction mass was quenched in an acid solution containing DM water (1000 mL) and 30% HCl (125 mL). The resulting mixture was filtered to remove the solid precipitate. The filtrate was then extracted with toluene (2×500 mL). The aqueous layer was treated with carbon (20 g) and the mixture was then filtered through a bed of diatomaceous earth. To the filtrate was added acetone (300 mL). The pH was adjusted to 8.5-9.5 using 25% aqueous ammonia solution (90-95 mL) to precipitate the product. The resulting suspension was filtered and the collected product was washed with acetone:water (1:9) to provide crude R-1-acetyl-5-(2-phenylsulphonylethenyl)-3-(N-methylpyrrolidin-2-ylmethyl)-1H-indole). (Yield: 120 g dry wt, 83.33%; Purity: 94.5%).

### Purification Method A:

Crude (R)-1-acetyl-5-(2-phenylsulphonylethenyl)-3-(N-methylpyrrolidin-2-ylmethyl)-1H-indole (100 g, 0.2366 mol) was stirred in methanol (600 mL) for 2.0 hr and then filtered to provide pure (R)-1-acetyl-5-(2-phenylsulphonylethenyl)-3-(N-methyl-pyrrolidin-2-ylmethyl)-1H-indole. (Yield 90 g, 90%; purity: 98.5%).

### Purification Method B:

Crude (R)-1-acetyl-5-(2-phenylsulphonylethenyl)-3-(N-methylpyrrolidin-2-ylmethyl)-1H-indole (100 g, 0.2366 mol) was dissolved in acetone (200 mL) at 40-45° C. To this solution was added water (250 mL) slowly to precipitate the product. The resulting slurry was stirred for 2 hrs after which the product was collected by filtration. The product was pure (R)-1-acetyl-5-(2-phenylsulphonylethenyl)-3-(N-methylpyrrolidin-2-ylmethyl)-1H-indole. (Yield: 79.4 g, 79.4%; Purity: 99.34%).

### Example 8

### Preparation of EPSIP

(R)-1-Acetyl-5-(2-phenylsulphonylethenyl)-3-(N-methylpyrrolidin-2-ylmethyl)-1H-indole (125 g, 0.2962 mol), methanol (1350 mL) and water (125 mL) were added to a 4 neck Round bottom flask at 20-25° C. with stirring. To this mixture was added potassium carbonate (14.5 g, 0.1025 mol). The resulting reaction mass was stirred at 40° C. for 1 hr. The reaction mass was then treated with Carbon (20 g). This mixture was filtered through a bed of diatomaceous earth. To the filtrate was added water (1350 mL) over period of 1 hr. The product precipitated and the mixture was stirred for 2 hrs. The product was collected by filtration and washed with methanol:water-1:1 to provide the crude (R)-5-(2-phenylsulphonylethenyl)-3-(N-methylpyrrolidin-2-ylmethyl)-1H-indole product. (Yield: 90 g 80%; Purity: 99.3%).

### Purification:

The crude (R)-5-(2-phenylsulphonylethenyl)-3-(N-methylpyrrolidin-2-ylmethyl)-1H-indole (115 g, 0.3022 mol) was dissolved in acetonitrile (345 mL) at 40-45° C. and water (345 mL) was added slowly to that solution. The product precipitated out during the water addition and the resulting slurry was stirred for 2 hrs. The product was collected by filtration to provide pure (R)-5-(2-phenylsulphonylethenyl)-3-(N-methyl-pyrrolidin-2-ylmethyl)-1H-indole. (Yield: 107 g dry, 93%; Purity: 99.75%).

### Example 9

### Preparation of EPSIP-MSA

Pure (R)-5-(2-phenylsulphonylethenyl)-3-(N-methylpyrrolidin-2-ylmethyl)-1H-indole (80 g, 0.2102 mol) was dissolved in acetone (400 mL) at 30-35° C. Methane sulphonic acid (21.2 g, 0.2207 mol) was added, and the product precipitated out. The resulting slurry was cooled to below 20° C. and stirred for 1.0 hrs. The slurry was filtered to provide pure (R)-5-(2-phenylsulphonylethenyl)-3-(N-methylpyrrolidin-2-ylmethyl)-1H-indole methane sulfonate salt. (Yield: 95.1 g dry, 95%; Purity: 99.91%.)

### Example 10

### Preparation of EPSIP-PTSA

EPSIP (25.0 g) and ethanol (100 mL) was combined and warmed to 40° C. to obtain a clear solution. PTSA.H₂O (12.5 g in 25 mL ethanol) was then added drop wise at 40-46° C. The resulting mixture was stirred with gradual cooling. The salt precipitated and was collected by filtration at 20-22° C. The collected salt was washed with ethanol (12.5 mL). The product was obtained in 82.58% yield (30.07 g dry wt.). Water: 0.37%; MP: 259.6-262.7° C.; Purity: 98.33%; ¹H NMR (DMSO): δ(ppm) 1.03-1.07 (t, 3H), 1.91 (s, 1H), 2.28 (s, 1H), 2.50 (s, 3H), 2.87-2.92 (s, 3H), 3.35 (s, 1H), 3.42-3.43 (s, 2H), 7.10-7.12 (d, 2H).

### Example 11

### Preparation of EPSIP-BSA

EPSIP (25.0 g) and ethanol (100 mL) were combined and warmed to 40° C. to obtain a clear solution. Benzene sulphonic acid (10.4 g in 25 mL ethanol) was then added drop wise at 40-43° C. The resulting mixture was stirred with gradual cooling to form a precipitate, which was collected by filtration at 20-22° C. The collected solid was washed with ethanol (12.5 mL). The product was obtained in 74.9% yield 26.62 g (dry wt.). (Water: 1.52%; MP: 190.1-192.6° C.; Purity: 98.33%)

### Example 12

### Preparation of EPSIP-TFA

EPSIP (5.0 g) and ethanol (25 mL) were combined and warmed to 35-40° C. to obtain a clear solution. TFA (1.5 g) was then added drop wise at 35-40° C. The resulting mixture was stirred with gradual cooling to form a precipitate, which was collected by filtration at 20-22° C. The collected precipitate was washed with ethanol (5 mL). The product was obtained in 67.12% yield (4.36 g dry wt.). Water: 2.47%; Purity: 98.33%; ¹H NMR (DMSO): δ(ppm) 1.55-1.56 (s, 1H), 1.94 (t, 3H), 1.99-2.01 (s, 1H), 2.50 (s, 3H), 2.88-2.91 (s, 3H), 2.95-3.05 (s, 1H), 3.36-3.62 (s, 2H), 7.36-7.7.43 (q, 4H), 7.52-7.57 (d, 2H), 7.64-7.67 (t, 3H), 7.70-7.73 (t, 3H), 7.77 (s, 1H), 7.91-7.93 (d, 2H), 8.02 (s, 1H), 11.36 (s, 1H); ¹³C NMR (DMSO): δ(ppm), 21.41, 29.93, 39.33, 39.54, 39.75, 39.96, 40.17, 40.38, 40.59, 56.33, 68.58, 112.78, 122.08, 122.21, 123.68, 124.24, 127.35, 130.07, 133.78, 138.33, 141.95, 144.73.

### Example 13

### Preparation of EPSIP Formate

EPSIP (2.0 g, 0.0052 mol) and acetonitrile (8 mL) were combined and warmed to 30-40° C. to obtain a clear solution. Formic acid (0.25 g, 0.0054 mol) was added at 30-40° C., followed by addition of ammonium formate (0.32 g, 0.0052). The resulting solution was cooled to 0-5° C. for 30 minutes. The formate salt of EPSIP was then collected by filtration and dried at 40-45° C. The product was obtained in 80% yield (1.8 g dry wt.). Purity: 96.0%; ¹H NMR (DMSO): δ(ppm) 1.55-1.59 (s, 1H), 1.64-1.71 (t, 3H), 1.78-1.82 (s, 1H), 2.42-2.47 (d, 2H), 2.50-2.52 (t, 3H), 2.64-2.70 (q, 1H), 2.79-2.86 (s, 1H), 3.13-3.22 (d, 2H), 7.35-7.39 (t, 3H), 7.42 (s, 1H), 7.47-7.50 (d, 1H), 7.63-7.66 (t, 2H), 7.68-7.72 (d, 2H), 7.76 (s, 1H), 7.91-7.94 (d, 2H), 7.97 (s, 1H), 8.31 (s, 1H). ¹³C NMR (DMSO): δ(ppm) 21.72, 28.11, 30.73, 39.29, 39.50, 39.71, 39.92, 40.13, 40.34, 40.55, 56.70, 66.96, 112.56, 113.04, 121.78, 122.29, 123.38, 123.92, 125.30, 127.34, 127.98, 130.02, 133.70, 138.21, 142.04, 144.92, 165.18.

### Example 14

### Preparation of EPSIP.Succinate

EPSIP (2.0 g, 0.0052 mol) and acetonitrile (8 mL) were combined and warmed to 45-50° C. to obtain a clear solution. Succinic acid (0.62 g, 0.0052 mol) was added at 45-50° C. and the resulting solution was slowly cooled at 20-25° C. for 30 minutes to precipitate the succinate salt of EPSIP. The precipitate was collected by filtration and dried at 40-45° C. The product was obtained in 77% yield (2.0 g dry wt.). Purity: 96.0%; ¹H NMR (DMSO): δ(ppm) 1.78-1.88 (t, 3H), 1.94-1.97 (t, 3H), 2.69-2.76 (m, 5H), 2.91-2.97 (q, 1H), 3.15-3.18 (t, 1H), 3.31-3.35 (dd, 2H), 3.56-3.60 (t, 1H), 6.87 (s, 1H), 6.91 (s, 1H), 7.17 (s, 1H), 7.32-7.34 (d, 1H), 7.39-7.41 (d, 1H), 7.55-7.55 (t, 1H), 7.57-7.59 (dd, 2H), 7.61-7.65 (s, 1H), 7.77 (s, 1H), 7.81 (s, 1H), 7.94 (t, 1H), 7.96-7.97 (s, 1H), 10.7-10.8 (s, 1H). ¹³C NMR (DMSO): δ(ppm) 21.48, 27.40, 30.71, 31.22, 39.42, 39.63, 39.83, 39.93, 40.04, 40.46, 40.67, 56.56, 67.77, 77.43, 77.76, 78.08, 11.87, 112.55, 121.22, 121.35, 123.08, 123.27, 124.95, 127.27, 127.36, 129.31, 133.12, 138.33, 141.49, 144.77, 176.57.

### Example 15

### Preparation of Eletriptan-PTSA

(R)-5-(2-Phenylsulphonylethenyl)-3-(N-methylpyrrolidin-2-ylmethyl)-1H-indole methane sulfonate salt (90 g, 0.1888 mol), acetone (450 mL) and water (90 mL) were combined to form a mixture and stirred to obtain a clear solution. The solution was cooled to 8-10° C. To the cooled solution was added methane sulphonic acid (11.47 g, 0.1194 mol). The resulting reaction mass was and added to a hydrogenation vessel and 10% palladium on carbon (18 g dry basis) was added. The reaction mixture was hydrogenated under an atmosphere of 2-5 kgs/cm² hydrogen gas for 2-3 hrs. When the reaction was complete, the reaction mixture was filtered to remove the catalyst. The filtrate was concentrated to distill out the acetone. After the distillation, the remaining filtrate was diluted with water (450 mL). MTBE (1350 mL) was added. The pH was adjusted to 9.0-9.5 using a 25% aqueous ammonia solution. The organic layer was separated and the aqueous layer was extracted with MTBE (450 mL). The organic (MTBE) portions were combined and concentrated to provide an oily suspension. To this suspension was added ethanol (90 mL) and the resulting mixture was concentrated to provide an oily mass. The oily mass was dissolved in ethanol (360 mL) at 40° C. and a solution of p-toluene sulphonic acid (36.6 g, 0.1925 mols dissolve in 90 mL ethanol) was added at 40-45° C. During the addition the product precipitated. The resulting slurry was cooled to 20° C. and stirred at that temperature for 1.0 hr. The product was collected by filtration and washed with ethanol (180 mL) and dried under reduce pressure at 60° C., to yield Eletriptan-PTSA salt. (Yield: 92.1 g dry, 89.76%. Purity: 99.8%).

### Example 16

### Preparation of Eletriptan-Base from Eletriptan-PTSA

Eletriptan-PTSA (45 g, 0.081 mol) was added to a mixture of DM water (180 mL) and MTBE (675 mL) at 20-25° C. The pH of mixture was adjusted to 10-10.5 using 25% aqueous ammonia. Two phases were formed and the layers were separated. The aqueous layer was washed with MTBE (225 mL) and layers were again separated. The two organic (MTBE) fractions were combined and washed with 10% brine solution. The organic layer was separated and concentrated at 40-45° C. under reduced pressure to obtain Eletriptan-base as an oil (Yield: 31.0 g, 99.83% 1; Purity: 99.75%).

### Example 17

### Preparation of Eletriptan-HBr from Eletriptan-Base

49% w/w Hydrobromic acid (2.13 g, 0.0262 mol) was added to a stirred solution of the oil obtained in Example 16 (5.0 g, 0.0130 mol) in acetone (50 mL) at room temperature. After 15 minutes, the reaction mixture was evaporated under reduced pressure to give a yellow liquid. The water remaining in this yellow liquid was then azeotropically removed using 2-propanol. The resulting cloudy, yellowish oil (7.2 g) was triturated with ether and then dissolved in hot 2-propanol (125 mL). This solution, on cooling, provided the title compound (5.95 g) as a pale yellow crystalline solid after filtration. The filtered solid was washed with 2-propanol and dried under vacuum. (purity 99.78%, yield 98.34%, Assay 10.9%, water by KF 3.41%) m.p. 170.8-172.9° C.

### Example 18

### Preparation of Eletriptan-HBr from Eletriptan-Base

49% w/w Hydrobromic acid (2.13 g, 0.0262 mol) was added over 1 hour to a stirred solution of the oil obtained in Example 16 (5.0 g, 0.0130 mol) in 1,2-dimethoxyethane (113 mL) at about 5° C. The cooling bath was removed and the resulting slurry was allowed to granulate by stirring at room temperature for an additional 18 hours. Filtration, followed by washing with 1,2-dimethoxyethane and drying in vacuum, afforded the product (3.95 g dry, Purity 97.79%, yield 65.28%, Assay 92.1%, water by KF 0.67%) as a solid, m.p. 115-116° C.

### Example 19

### Comparative Preparation of Acetyl EPSIP According to Example 1 of WO 2005/007649

(a) (R)-5-Bromo-3-(N-methylpyrrolidine-2-ylmethyl)-1H-indole (100 g), acetonitrile (147 mL), TEA (44.92 g) and acetic anhydride (44.92 g) were charged to a dry glass lined vessel. The resulting mixture was heated to reflux and maintained at this temperature for 4.5 hours.

(b) A mixture of acetonitrile (147 mL), palladium acetate (4.88 g) and tri-o-tolylphosphine (23.4 g) was stirred for 1 hour. Phenyl vinyl sulphone (62.5 g), TEA (35.93 g) and the solution prepared in part (a) were added and the resulting mixture was heated to reflux for 7.5 hours. The reaction mixture was cooled and a solution of 74.2 g of concentrated HCl in 469 mL water was added over 4 hours. The resulting mixture was filtered to remove spent catalyst. Water (1172 mL) and 117.1 mL of 50% w/w aqueous sodium hydroxide solution were added to the filtrate to produce a sticky mass. The product did not precipitate after 4.5 hours. The reaction mixture was stirred for 14-16 hour, after which the product was still not precipitated. The water was decanted to provide the crude (R)-1-acetyl-5-(2-phenylsulphonylethenyl)-3-(N-methylpyrrolidin-2-ylmethyl)-1H-indole) as a dark brown, sticky mass. This crude sticky product was added to 207 g of acetone. This mixture was heated to 60° C. On reaching this temperature 317 g of water was added over 2 hours whilst simultaneously cooling the mixture to ambient temperature. The batch was then granulated for 2 hours. The product did not solidify, and the dark brown sticky mass was still observed after 16 hours of stirring.

(R)-1-acetyl-5-(2-phenylsulphonylethenyl)-3-(N-methylpyrrolidin-2-ylmethyl)-1H-indole (“Acetyl EPSIP”): 58.32% (RT=117.29); (Methylsulfonyl)benzene (“EPS”): 10.47% (RT=7.26); tri-O-tolyl phosphine (“TOTP”): 17.98% (RT=32.19); 1,2-bis(phenylsulfonyl)ethane) (“EPS”) Dimer: 1.0% (RT=19.85); 3-[(R)-1-methyl-pyrrolidin-2-ylmethyl]-1H-indole (Des bromo BIP) (“DBBIP”): 0.81% (RT=5.13).

### Example 20

### Comparative Preparation of EPSIP According to Example 2 of WO 2005/007649

Methanol (936.8 g) and (R)-1-acetyl-5-(2-phenylsulphonylethenyl)-3-(N-methylpyrrolidin-2-ylmethyl)-1H-indole (109.3 g dry equivalent of the sticky product of Example 1) were charged to a vessel. The resulting mixture was stirred for 5 minutes. Potassium carbonate (12.6 g) was added and this mixture was stirred at room temperature for 30 minutes. The resulting mixture was then warmed to 35° C. and Eno carbon (16.5 g) and water (333.5 g) were added. The resulting mixture was filtered. The filtrate was diluted by the addition of water (1845 g, added over a half hour), and granulated for 2 hours at room temperature. The product did not solidify, and dark brown lumps were observed after 16 hours stirring at room temperature. Filtration of the mixture provided crude (R)-5-(2-phenylsulphonylethenyl)-3-(N-methylpyrrolidin-2-ylmethyl)-1H-indole (108 g wet, Purity 72.82%). EPSIP: 72.82% (RT=16.02); Acetyl EPSIP: 0.73% (RT=16.89); TOTP: 1.40% (RT=32.24); EPS Dimer: 0.03% (RT=19.58); DBBIP: 2.29% (RT=5.06).

### Example 21

### Comparative Preparation of EPSIP According to Example 3 of WO 2005/007649

A mixture of acetonitrile (169 g) and crude (R)-5-(2-phenylsulphonylethenyl)-3-(N-methylpyrrolidin-2-ylmethyl)-1H-indole (85.85 g dry equivalent, product of the process of Example 2) was warmed to 55° C. Water (129.5 mL) was added, and the resulting mixture was cooled to 20° C. and granulated for 2 hours at that temperature. Pure (R)-5-(2-phenylsulphonylethenyl)-3-(N-methylpyrrolidin-2-ylmethyl)-1H-indole (90.5 g wet, equivalent of 70.70 g dry, Purity 88.45%,) was recovered by filtration. EPSIP: 88.45% (RT=16.02); Acetyl EPSIP: 0.32% (RT=16.89); TOTP: 1.22% (RT=32.23); DBBIP: 0.91% (RT=5.09).

### Example 22

### Comparative Preparation of EPSIP According to Example 4 of WO 2005/007649

Acetone (167 g) and (R)-5-(2-phenylsulphonylethenyl)-3-(N-methylpyrrolidin-2-ylmethyl)-1H-indole (70.5 g dry equivalent, recrystallized product of Example 3) were charged to a vessel. The mixture was warmed to 55° C. Water (222 g) was added and the resulting mixture was cooled to 20° C. and granulated for 2 hours. Recrystallized (R)-5-(2-phenylsulphonylethenyl)-3-(N-methylpyrrolidin-2-ylmethyl)-1H-indole (87 g wet, 53.0 g dry equivalent, yield 74.96%, Purity 88.06%) was isolated by filtration. EPSIP: 88.06% (RT=16.05); Acetyl EPSIP: 0.38% (RT=16.90); TOTP: 0.58% (RT=32.21); DBBIP: 0.53% (RT=5.13).

### Example 23

### Comparative Preparation of ELT According to Example 5 of WO 2005/007649

(R)-5-(2-Phenylsulphonylethenyl)-3-(N-methylpyrrolidin-2-ylmethyl)-1H-indole (50 g dry equivalent) and acetone (296 g) were charged to a dry, glass lined vessel. De-ionized water (75 mL), acetone (59.2 g), methanesulphonic acid (13.75 g) and a slurry of 10% palladium on carbon (11.2 g 50% wet) in de-ionized water (50 mL) were added. The resulting mixture was hydrogenated under one atmosphere of hydrogen gas. The reaction was not completed after 3.0 hr. The reaction mixture was filtered and fresh 10% palladium on carbon (11.2 g 50% wet) was added, and the reaction was continued in an autoclave under 5.0 kg hydrogen gas pressure for 26 hrs. Reaction monitoring showed only 52% product formation by HPLC. The reaction mixture was filtered and again, fresh catalyst 10% palladium on carbon (11.2 g 50% wet) was added and the reaction was continued in an autoclave under 5.0 kg hydrogen gas pressure at 20-25° C. temperature for 40 hrs. Reaction monitoring showed 68.10% product formation by HPLC. The hydrogenation reaction was stopped. ELT: 52.60% (RT=13.61); EPSIP: 36.85% (RT=15.95)

### Example 24

### Preparation of BIP.Potassium

BIP (5.0 g, 0.017 mol) and ethanol (10 mL) were combined and warmed to 35° C. to obtain a clear solution. Potassium hydroxide (1.12 g in 5 mL ethanol) was added drop wise at 35-40° C. The resulting mixture was stirred with gradual cooling and the salt that precipitated was collected by filtration at 18-20° C., and was washed with ethanol (5 mL). Yield: 28.3%; Water: 2.05%; Melting range: 128.5-129.8° C.; Purity: 99.23%; ¹H-NMR (400 MHz, DMSO-d6): δ(ppm) 2.04-2.15 (q, 2H), 2.43-2.50 (dd, 3H), 2.92-3.01 (dd, 3H), 7.14-7.19 (t, 2H), 7.29-7.31 (d, 1H), 7.63 (s, 1H); ¹³C NMR (100 MHz, DMSO-d6): δ(ppm) 21.96, 29.43, 31.26, 39.22, 39.43, 39.63, 39.84, 40.05, 40.26, 40.47, 40.93, 57.38, 66.55, 111.27, 112.47, 113.82, 121.04, 123.63, 125.14, 129.85, 135.22.

### Example 25

### Preparation of BIP Oxalate

BIP (150.0 g, 0.5115 mol) and methanol (900 mL) were combined and warmed to 40-45° C. to obtain a clear solution. Oxalic acid dihydrate (64.5 g, 0.5115 mol) in 300 mL methanol was added drop wise at 40-45° C. The resulting mixture was stirred with gradual cooling and the salt precipitate that was formed was filtered at 20-25° C. The filtered solid was washed with methanol (150 mL). Dry wt: 175 g; Yield: 89.23%; Water: 0.05%; Melting range: 121.8-123.4° C.; Purity: 97.55% (as a bip salt); ¹H-NMR (400 MHz, DMSO-d6) δ(ppm) 2.05-2.11 (q, 2H), 2.43-2.50 (dd, 3H), 2.92-2.98 (dd, 3H), 3.00-3.01 (s, 1H), 7.10-7.16 (d, 1H), 7.19 (s, 1H), 7.29-7.31 (d, 1H), 7.63-7.64 (s, 1H), 11.01 (s, 1H); ¹³C NMR (100 MHz, DMSO-d6) δ(ppm) 21.96, 29.43, 31.27, 39.22, 39.43, 39.64, 39.85, 40.06, 40.27, 40.48, 40.93, 57.38, 66.55, 111.28, 112.49, 113.81, 121.04, 123.64, 125.13, 129.85, 135.22.

### Example 26

### Preparation of BIP from BIP Oxalate

BIP oxalate salt (170 g, 0.4435 mol) and DM water (1500 mL) was stirred at 15° C. The pH was adjusted to 7.8 using aqueous sodium carbonate (70.5 g, 0.6650 mol) in 200 mL DM water. Toluene (1000 mL) was added and stirred at 30-35° C. for 30 minutes. The layers were separated and the aqueous layer was washed with toluene (500 mL). The layers were again separated. The two toluene layer were combined and washed with 10% w/v aqueous sodium carbonate. The layers were separated and the toluene layer was distilled under reduce pressure at 50-55° C. to 70% (1050 mL). The distilled mass was gradually cooled to 5-10° C. with stirring. A solid product precipitated and was collected by filtration and washed, first with cold toluene (100 mL) and then with n-heptane (100 mL). Dry. Wt: 117.0 g; Yield: 90.5%; Purity: 99.12% (as a Bip).

### Example 27

### Preparation of BIP-Succinate

BIP (10.0 g, 0.0341 mol) and methanol (50 mL) were combined and warmed to 40-45° C. to obtain a clear solution. Succinic Acid (4.0 g, 0.0341 mols) was added drop wise at 40-45° C. The resulting mixture was stirred with gradual cooling. The precipitated BIP Succinate salt was collected by filtration at 5-10° C., and washed with cooled methanol (20 mL). Dry wt: 13.1 g; Yield: 93%; Water: 0.26%; Purity: 99.82%; Melting Range: 171.2-174.5°; ¹H-NMR (400 MHz, DMSO-d6) δ(ppm) 2.35-2.40 (s, 9H), 2.50 (s, 1H), 2.61-2.71 (t, 2H), 2.83 (s, 2H), 2.99-3.13 (d, 2H), 3.16 (s, 1H), 7.16-7.18 (d, 1H), 7.25 (s, 1H), 7.31-7.33 (d, 1H), 7.70 (s, 1H), 11.10 (s, 1H); ¹³C NMR (100 MHz, DMSO-d6) δ(ppm) 21.68, 27.96, 30.60, 30.82, 39.22, 39.43, 39.64, 39.85, 40.06, 40.27, 40.48, 56.79, 67.18, 111.20, 111.51, 113.93, 121.04, 123.88, 125.54, 129.53, 135.28, 174.97.

### Example 28

### Preparation of BIP-Fumarate

BIP (10.0 g, 0.0341 mol) and methanol (50 mL) were combined and warmed to 40-45° C. to obtain a clear solution. Fumaric acid (4.0 g, 0.0341 mol) was added drop wise at 40-45° C. The resulting mixture was stirred with gradual cooling and the precipitated Bip succinate salt was collected by filtration at 5-10° C., and washed with cold methanol (20 mL). Dry wt: 12.6 g; Yield: 90.0%; Water: 0.27%; Purity: 99.82%; Melting Range: 198.6-202.2° C.; ¹H-NMR (400 MHz, DMSO-d6) δ(ppm) 2.68 (s, 6H), 2.79-2.85 (d, 4H), 3.16-3.25 (d, 3H), 6.53 (s, 3H), 7.07-7.19 (d, 2H), 7.30 (s, 1H), 7.32-7.34 (d, 2H), 7.76 (s, 1H), 11.21 (s, 1H); ¹³C NMR (100 MHz, DMSO-d6) δ(ppm) 21.43, 26.61, 30.19, 39.21, 39.33, 39.42, 39.63, 39.84, 40.05, 40.26, 40.47, 56.01, 67.42, 110.41, 111.66, 114.00, 121.04, 124.02, 125.83, 129.27, 135.33, 135.50, 168.36.

### Example 29

### Preparation of BIP from BIP Oxalate

BIP oxalate salt (170 g, 0.4435 mol) and DM water (1500 mL) was stirred at 15° C. The pH was adjusted to 7.8 using aqueous sodium carbonate (70.5 g, 0.6650 mol) in 200 mL DM water. Toluene (1000 mL) was added and the resulting mixture was stirred at 30-35° C. for 30 minutes. The aqueous layers was separated and further washed with toluene (500 mL). The two toluene fractions were combined and washed with 10% w/v aqueous sodium carbonate. The layers were separated and the toluene layer was distilled under reduced pressure at 50-55° C. to 70% (1050 mL). The distilled mass was gradually cooled to 5-10° C. temperature with stirring. The solid product precipitated and was filtered and washed, first with cold toluene (100 mL) and then with n-heptane (100 mL). Dry. Wt: 117.0 g; Yield: 90.5%; Purity: 99.12% (as a Bip).

### Example 30

### Preparation of EPSIP from BIP

DMF was added to a 1.0 lis cap four neck Round bottom flask equipped with a gas purging tube and a water-cooled condenser. Argon gas was purged through dip pipe for 30 minutes. Palladium acetate (1.53 g, 0.0068 mol) and tri o-tolylphosphine (8.3 g, 00272 mol) were added and the resulting mixture was stirred for 30 minutes. To this mixture were added diisopropylethylamine (26.44 g, 0.2046 mol), BIP (50 g, 0.1705 mol) and phenyl vinyl sulphone (31.55 g, 0.1675 mol). The resulting mixture was heated to 95-100° C. and stirred at that temperature for 90-120 minutes. The mixture was cooled to 20-25° C. Dilute HCl (Mixture of 600 mL water and 32 mL Conc. HCl) was added. To this was added diatomaceous earth (25 g) and the mixture was stirred for 30 minutes and then filtered. The filtrate was extracted with toluene (2×150 mL). Acetone (150 mL) was added to the aqueous fraction and the pH was adjusted to 9.5-10 with ammonia solution and then stirred for 2.0-4.0 hours. A solid precipitate formed and was collected by filtration and washed with water (400 mL). Wet product: 76-77 g; Dry product: 51-52 g; yield: 79.62%

### Example 31

### Purification of Crude EPSIP

The crude EPSIP (50 g) product of example 30 was combined with acetonitrile (150 mL) and heated to 45-50° C. to form a clear solution. To this was added water (150 mL) over a period of 90-120 minutes. This mixture was stirred for 5.0-7.0 hours to complete the precipitation. The precipitate was filtered and the wet product was washed with a mixture of acetonitrile and water (50:50, 50 mL) and dried at less than 40° C. to obtain dry EPSIP. Dry weight: 43.1 g; Purity: 95.56 g; % m/c: 5.6%; yield: 86.2%

### Example 32

### Preparation of Eletriptan Para Toluene Sulphonate

The pure EPSIP (25 g, 0.065 mol) prepared from example 31 was dissolved in acetone (250 mL) and combined with water (25 mL) and methane sulphonic acid (10.09 g, 0.105 mol). To this mixture was added 10% Pd/C 50% wet (12.5 g) and the resulting mixture was hydrogenated at 1 Bar hydrogen atmosphere for 3.0-4.0 hours. The reaction mixture was filtered and the filtrate was distilled under reduced pressure. To the concentrate was added water (25 mL), and the pH was adjusted to 9-10 with ammonia solution. This mixture was extracted twice with MTBE (500+125 mL). The combined organic fractions were washed with brine (100 mL) and concentrated to get oily residue. To the residue was added ethanol (100 mL) to form a clear solution. To this solution was added a solution of PTSA (50 g PTSA dissolved in ethanol, 25 mL) over 60-90 minutes at 45-50° C. This mixture was stirred for 4-5 hrs time. The resulting slurry was filtered and the filter cake was washed with ethanol (25 mL), and dried at 40-45° C. under reduced pressure. Dry weight 100 g; Yield: 67.58%; Purity 98.42%.

### Example 33

### Preparation of 5-Bromo-3-((R)-1-methyl-pyrrolidin-2-ylmethyl)-1H-indole (BIP) According to U.S. Pat. No. 5,545,644, Example 27

| BIPCAM | 3.64 g (8.52 mmol) |
|---|---|
| LAH | 1.26 g (33.2 mmol 3.9 eq) |
| THF | 122.5 |

A solution of (R)-2-(5-bromo-1H-indole-3-carbonyl)-pyrrolidine-1-carboxylic acid benzyl ester (BIPCAM) (3.64 g, 8.55 mmol) in dry THF (70 mL) was added drop-wise to a stirred suspension of lithium aluminum hydride (LAH)(0.945 g, 24.9 mmol) in dry THF (52.5 mL) at room temperature under an atmosphere of dry nitrogen. The mixture was heated under reflux with stirring for 18 h and then cooled. Additional LAH (175 mg, 4.61 mmol) was added and refluxing was continued for an additional 3 h. The mixture was again cooled, and LAH (140 mg, 3.69 mmol) was added, and refluxing was continued for a further 18 h. The mixture was cooled and H₂O (1.54 mL) was carefully added with stirring, followed by 20% aqueous NaOH (1.54 mL), followed by more H₂O (4.66 mL). The mixture was stirred for 30 min, then diluted with ethyl acetate (50 mL) and filtered through diatomaceous earth. The filtrate was washed with H₂O (50 mL), and brine (50 mL), and then dried with Na₂SO₄. Evaporation of the solvent gave an oil (3.6 g) which was chromatographed on silica gel, eluted with dichloromethane/ethanol/conc. aqueous ammonia (90:10:0.5) to obtain the title compound (1.18 g, 47%) as a light yellow oil. The product precipitated from dichloromethane/hexane (920 mg, 37%).

### HPLC Analysis:

### Crude Reaction Mixture (Norm %):

| | |
|---|---|
| benzyl alcohol | 26.25% |
| 3-[(R)-1-methyl-pyrrolidin-2-ylmethyl]-1H-indole | 1.03% |
| (“des-Bromo-BIP”) | |
| (R) (5-bromo-(1H-indol-3yl) (1-methyl-pyrrolidin-2yl) methanol | 10.75% |
| (“OH-BIP”) | |
| 5-bromo-(1H-indol-3yl) (1-methyl-pyrrolidin-2yl) methanone | 0.14% |
| (“keto-BIP”) | |
| BIP | 57.02% |

### Purified Product (Norm %):

| | |
|---|---|
| des-Bromo-BIP | 0.52% |
| OH-BIP | 0.14% |
| keto-BIP | 0.11% |
| BIP | 97.8% |

We claim:

1. A salt of (R)-5-(2-phenylsulphonylethenyl)-3-(N-methylpyrrolidin-2-ylmethyl)-1H-indole of the formula: 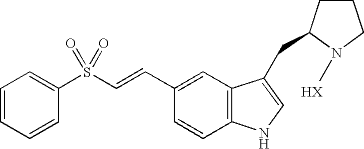
wherein HX is an acid selected from para-toluene sulfonic acid, benzene sulphonic acid, trifluoroacetic acid, methane sulphonic acid, formic acid and succinic acid.

2. A process for preparing a salt of (R)-5-(2-phenylsulphonylethenyl)-3-(N-methyl-pyrrolidin-2-ylmethyl)-1H-indole selected from a para-toluene sulfonic acid salt, a benzene sulphonic acid salt, a trifluoroacetic acid salt, a methane sulphonic acid salt, a formic acid salt, and a succinic acid salt, said process comprising reacting (R)-5-(2-phenylsulphonylethenyl)-3-(N-methylpyrrolidin-2-ylmethyl)-1H-indole with an acid selected from para-toluene sulfonic acid, benzene sulphonic acid, trifluoroacetic acid, methane sulphonic acid, formic acid and succinic acid.

3. The process of claim 2, wherein the reaction comprises:
a) dissolving (R)-5-(2-phenylsulphonylethenyl)-3-(N-methylpyrrolidin-2-ylmethyl)-1H-indole in a solvent to obtain a solution,
b) combining said solution with an acid selected from para-toluene sulfonic acid, benzene sulphonic acid, trifluoroacetic acid, methane sulphonic acid, formic acid, and succinic acid, and
c) precipitating the (R)-5-(2-phenylsulphonylethenyl)-3-(N-methylpyrrolidin-2-ylmethyl)-1H-indole salt to obtain a suspension.

4. The process of claim 3, wherein the (R)-5-(2-phenylsulphonylethenyl)-3-(N-methyl-pyrrolidin-2-ylmethyl)-1H-indole is dissolved in an organic solvent selected from a ketone, an alcohol, a nitrile and combinations thereof.

5. The process of claim 4, wherein the ketone is a C₁₋₄ ketone, the nitrile is a C₁₋₃ nitrile, and the alcohol is a C₁₋₄ alcohol.

6. The process of claim 5, wherein the C₁₋₄ ketone is acetone, the C₁₋₃ nitrile is acetonitrile, and the C₁₋₄ alcohol is methanol or ethanol.

7. The process of claim 3, wherein the precipitated (R)-5-(2-phenylsulphonylethenyl)-3-(N-methylpyrrolidin-2-ylmethyl)-1H-indole salt is recovered from the suspension.

8. A process for preparing Eletriptan and salts thereof comprising preparing (R)-5-(2-phenylsulphonylethenyl)-3-(N-methylpyrrolidin-2-ylmethyl)-1H-indole salts according to claim 3, and converting them to an Eletriptan salt.

9. A para-toluene sulphonic acid salt of Eletriptan of formula VII:

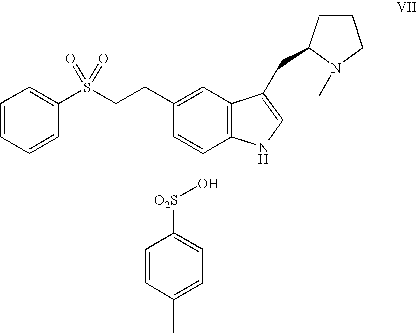

10. A process for preparing a para-toluene sulphonic acid salt of Eletriptan comprising reacting Eletriptan with para-toluene sulphonic acid.

11. A process for the preparation of eletriptan HBr comprising preparing a para-toluene sulphonic acid salt of Eletriptan according to claim 11, and converting it to eletriptan HBr.

12. A salt of 5-bromo-3-[(R)-1-methyl-pyrrolidin-2-ylmethyl]-1H-indole of the formula: 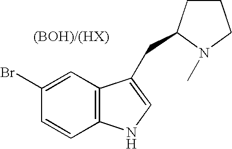
wherein B is potassium, and HX is an acid selected from oxalic acid, succinic acid, and fumaric acid.

13. A process for the preparation of a 5-bromo-3-[(R)-1-methyl-pyrrolidin-2-ylmethyl]-1H-indole salt comprising reacting 5-bromo-3-[(R)-1-methyl-pyrrolidin-2-ylmethyl]-1H-indole with an acid selected from oxalic acid, succinic acid and fumaric acid or with potassium hydroxide, wherein the obtained salt is an oxalate, succinate, fumarate or potassium salt.

14. The process of claim 13, wherein the reaction comprises
a) dissolving 5-bromo-3-[(R)-1-methyl-pyrrolidin-2-ylmethyl]-1H-indole to obtain a solution, and
b) combining said solution with an acid selected from oxalic acid, succinic acid, and fumaric acid or potassium hydroxide to obtain a suspension.

15. The process of claim 14, wherein 5-bromo-3-[(R)-1-methyl-pyrrolidin-2-ylmethyl]-1H-indole is dissolved in an alcohol.

16. The process of claim 15, wherein the alcohol is C₁₋₄ alcohol.

17. The process of claim 16, wherein the C₁₋₄ alcohol is methanol or ethanol.

18. The process of claim 14, wherein the precipitated 5-bromo-3-[(R)-1-methyl-pyrrolidin-2-ylmethyl]-1H-indole salt is recovered from the suspension.

19. A process for the preparation of Eletriptan HBr comprising preparing a 5-bromo-3-[(R)-1-methyl-pyrrolidin-2-ylmethyl]-1H-indole salt according to the process of claim 14, and converting it to eletriptan and salts thereof.
